(19) United States
(12) Patent Application Publication
(10) Pub. No.: US 2018/0134651 A1
(43) Pub. Date: May 17, 2018

(54) METHOD OF PURIFYING TEREPHTHALATE BASED ESTER COMPOUNDS

(71) Applicant: OCI COMPANY LTD., Seoul (KR)

(72) Inventors: Seong Hwan LEE, Seongnam-si (KR); Sang Hoon KIM, Seongnam-si (KR); Kyung Yeol KIM, Seongnam-si (KR); Yong Il KIM, Seongnam-si (KR)

(21) Appl. No.: 15/718,587

(22) Filed: Sep. 28, 2017

(30) Foreign Application Priority Data
Nov. 11, 2016 (KR) 10-2016-0150294

(51) Int. Cl. C07C 67/56; C07C 67/60; C07C 67/54; B01D 37/02

(52) U.S. Cl. CPC C07C 67/56; B01D 37/02; C07C 67/54; C07C 67/60

(57) ABSTRACT

Disclosed is a method of purifying terephthalate-based ester compounds, including: adding a base to a resultant reaction mixture comprising terephthalate-based ester compounds synthesized by esterification of terephthalic acid and alcohol so as to neutralize unreacted acids; applying steam to the resultant reaction mixture which has been neutralized; adding a filter aid to the resultant reaction mixture which has been steamed prior to filtration through a filter; and filtering through a filter the resultant reaction mixture to which the filter aid has been added, wherein the filter aid comprises diatomite particles having an average particle size of 50 μm to 90 μm.

### CROSS-REFERENCE TO RELATED APPLICATIONS

This application claims the priority of Korean Patent Application No. 10-2016-0150294 filed on Nov. 11, 2016, in the Korean Intellectual Property Office, the disclosure of which is hereby incorporated by reference in its entirety.

### BACKGROUND

### 1. Technical Field

The present disclosure relates to a method of purifying terephthalate based ester compounds.

### 2. Description of the Related Art

In general, the preparation of plasticizer largely includes reaction process, de-alcoholizing process for removing an excess of alcohol, purification process for removing impurities, or filtration process. Acidic catalyst such as PTSA (p-toluene sulfonic acid) or metallic catalyst such as TIPT (tetraisopropyl titanate) may be used as reaction catalyst. The purification process is a final stage for the production of finished plasticizer in the preparation of plasticizer. During this process, unreacted materials, metallic ions, neutralized salts, catalytic salts, and the like are removed. In some cases, rinsing process for several times of water washing may be applied to remove impurities generated after neutralization process, prior to the purification process. This may cause problems of treating a large amount of wastewater and increased process time.

### SUMMARY

It is an aspect of the present disclosure to provide a method of purifying terephthalate-based ester compounds which can perform at an improved filtration rate without resorting to a rinsing process and prevent speed reduction and filter clogging. spIn one embodiment of the present disclosure, there is provided a method of purifying terephthalate-based ester compounds which includes adding a base to a resultant reaction mixture comprising terephthalate-based ester compounds synthesized by esterification of terephthalic acid and alcohol so as to neutralize unreacted acids; applying steam to the resultant reaction mixture which has been neutralized; adding a filter aid to the resultant reaction mixture which has been steamed prior to filtration through a filter; and filtering through a filter the resultant reaction mixture to which the filter aid has been added, wherein the filter aid comprises diatomite particles having an average particle size of 50 μm to 90 μm.

The alcohol may be an alkanol having a linear or branched (C₄-C₁₃) alkyl group.

The alcohol may include at least one selected from the group consisting of butanol, 2-ethylhexanol, isononyl alcohol, isodecyl alcohol, propyl heptanol, and combinations thereof.

The base may include at least one selected from the group consisting of sodium hydroxide (NaOH), sodium carbonate (Na₂CO₃), sodium bicarbonate (NaHCO₃), and combinations thereof.

The steam may be at a temperature of 140 to 200° C. and under a pressure of 2 bar to 10 bar.

The diatomite may have a pH of 7 to 10 and include 90 to 100 parts by weight of SiO₂, 1 to 10 parts by weight of Al₂O₃, 1 to 10 parts by weight of Fe₂O₃, 0.1 to 1 part by weight of CaO, 0.1 to 1 part by weight of MgO, and water.

The diatomite may be added in an amount of 1 to 10 parts by weight based on 100 parts by weight of the resultant reaction mixture which has been steamed.

The filter may be a filter membrane on which a precoat layer containing diatomite particles is formed.

The method removes neutralized salts of unreacted terephthalates.

The method may be a rinsing-free process.

The method of purifying terephthalate-based ester compounds does not require rinsing process and can reduce filtration time and thus shorten processing time by using diatomite having similar size to neutralized salt as a filter aid in filtration process.

### BRIEF DESCRIPTION OF THE DRAWINGS

FIG. 1 is a schematic diagram illustrating a method of purifying terephthalate-based ester compounds according to one embodiment of the present invention.

### DETAILED DESCRIPTION

Hereinafter, exemplary embodiments of the present disclosure will be described in detail with reference to the accompanying drawings, so that they can readily be practiced by those skilled in the art to which the present disclosure pertains. The present disclosure may be embodied in many different forms and is not limited to the embodiments described herein.

In one embodiment of the present disclosure, there is provided a method of purifying terephthalate-based esters including:

adding a base to a resultant reaction mixture comprising terephthalate-based ester compounds synthesized by esterification of terephthalic acid and alcohol so as to neutralize unreacted acids;

applying steam to the resultant reaction mixture which has been neutralized;

adding a filter aid to the resultant reaction mixture which has been steamed prior to filtration through a filter; and

filtering through a filter the resultant reaction mixture to which the filter aid has been added,

wherein the filter aid comprises diatomite particles having an average particle size of 50 μm to 90 μm.

The resultant reaction mixture comprising terephthalate-based ester compounds synthesized by esterification of terephthalic acid and alcohol may be obtained in the form of a mixture of unreacted materials, metallic ions, neutralized salts and catalytic salts together with the synthesized ester compounds, such that the resultant reaction mixture may contain impurities. Typically, the impurities may be removed from the resultant reaction mixture by a rinsing process. Since the method of purifying terephthalate-based ester compounds are free of rinsing process, facility investment required for the rinsing process is not required, and a large amount of wastewater generated by the rinsing process is not produced. Therefore, additional facility investment expenditures and wastewater treatment costs can be saved.

Hereinafter, each step of the method of purifying terephthalate-based ester compounds will be described in detail.

The method can be effectively applied to terephthalate-based ester compounds produced by an ester exchange reaction of terephthalic acid and an alcohol.

The terephthalate-based ester compounds may be used, for example, as a plasticizer, but not limited thereto.

For example, when the terephthalate-based ester compounds are used as a plasticizer, if the content of impurities added in the terephthalate-based ester compounds is high, the terephthalate-based ester compounds are inhibited from manifesting various physical properties required as a plasticizer. Therefore, in order to exhibit excellent physical properties when the terephthalate-based ester compound is applied as a plasticizer, it is necessary to reduce the content of impurities mixed in the synthesis of the terephthalate-based ester compounds.

The alcohol may be an aliphatic alcohol, specifically, an alkanol having a linear or branched (C₄-C₁₃) alkyl group.

The alcohol may include at least one selected from the group consisting of butanol, 2-ethylhexanol, isononyl alcohol, isodecyl alcohol, propyl heptanol, and combinations thereof.

The resultant reaction mixture contains impurities as described above, particularly, unreacted acids, which must be necessarily neutralized. Thus, base may be added to the resultant reaction mixture to neutralize the unreacted acids.

The base, for example, may include, but is not limited to, at least one selected from the group consisting of sodium hydroxide (NaOH), sodium carbonate (Na₂CO₃), sodium bicarbonate (NaHCO₃), and combinations thereof.

The base may be added in an amount of 0.1 to 1 part by weight based on 100 parts by weight of the resultant reaction mixture which has been neutralized. Within the above range of contents, the pH of the resultant reaction mixture can be suitably controlled.

Specifically, the resultant reaction mixture which has been neutralized may have a pH of 7 to 10.

When high-temperature and high-pressure steam is applied to the resultant reaction mixture having been subjected to neutralization treatment, alcohol is removed by azeotropic distillation together with steam. In addition to removing the unreacted alcohol by the steam treatment step, Ti-based catalyst may react with water and be extracted into solid state TiO₂, and the TiO₂ may be removed by a subsequent filtration step.

The high-temperature and high-pressure steam may be at a temperature of 140 to 200° C. and under a pressure of 2 bar to 10 bar.

Subsequently, a filter aid is added to the resultant reaction mixture which has been steamed.

The filter aid may use diatomite particles having an average particle size of 50 μm to 90 μm.

When the diatomite particles are used as a filter aid, filtration rate is improved in a subsequent filtration step, the filter is protected, and in particular, the neutralized salts generated in the previously performed neutralization process can be efficiently removed.

Diatomite is a kind of fossil soil that is a collection of debris from single-celled plants called diatoms, deposited on a sea-bed and a bottom of lake. Diatoms have the ability to extract silica from underwater environments to form their own rigid skeletal structure, wherein diatomaceous sedimentary rocks formed by sedimentation of diatoms skeleton under the influence of diagenesis are mostly composed of amorphous silica and are produced in a large scale aggregation state. Diatomite is classified as fresh water type and sea water type depending on the places where it is deposited, and also shows different physical properties. The diatomite used as the filter aid is a porous structure containing silica as a main component and has a melting point of 1400° C. or more and is stable to general chemical action.

Specifically, the diatomite may have a pH value of 7 to 10.

Specifically, the diatomite may include 90 to 100 parts by weight of SiO₂, 1 to 10 parts by weight of Al₂O₃, 1 to 10 parts by weight of Fe₂O₃, 0.1 to 1 part by weight of CaO, 0.1 to 1 part by weight of MgO, and a small quantity of water.

The diatomite particles may have an average particle diameter of 50 μm to 90 μm, and the filter aid having a particle size in the above range is effective for removing neutralized salts similar in size. When the filter aid forms a filtering layer, intergranular pores are generated, which are determined by the particle size. Since the size of the pores determines the filtration efficiency, the efficiency of neutralized salt removal can be improved by using a silicate having a size similar to that of the neutralized salts. The size of the neutralized salts of unreacted terephthalate generated during the synthesis of the terephthalate-based ester compounds is similar to that of the diatomite particles. Therefore, the filtration rate can be improved by using the diatomite having a size similar to the neutralized salt of unreacted terephthalate as a filter aid.

The method of purifying the terephthalate-based ester compounds can effectively remove the impurities including the neutralized salts of unreacted terephthalates to improve the filtration rate.

Generally, in the case of rinsing-free process, since the impurities are not removed by water, it is necessary to remove fine particles such as neutralized salts of unreacted terephthalates, inorganic salts and organic salts through a filtration filter, which may cause process speed of filter to slow or filter clogging. Impurities such as neutralized salts of unreacted terephthalates, inorganic salts and organic salts have a very small crystal grain sizes and easily form a film on the filter surface, which in turn easily cause problems of filter clogging.

Since the method of purifying terephthalate-based ester compounds, even as a rinsing-free process, can effectively remove the neutralized salts by using the filter aid, the method of purifying terephthalate-based ester compounds is able to improve the filtration rate and efficiency of the filter. Since the method of purifying terephthalate-based ester compounds possibly shortens the process time as it improves the filtration rate, it possibly reduces the production process time of the terephthalate-based ester compounds.

The diatomite particles added to the resultant reaction mixture which has been steamed may be added in an amount of 1 to 10 parts by weight based on 100 parts by weight of the steam-treated resultant reaction mixture. The effect of removing the impurities can be further improved by adding the diatomite particles within the above amount.

The filter may include diatomite. Specifically, the filter may be a filter membrane on which a precoat layer containing diatomite particles is formed.

The detailed description of the diatomite particles contained in the precoat layer may be the same as described for the diatomite used as a filter aid.

The filter membrane may be that formed of a known material such as polypropylene, polytetrafluoroethylene (PTFE), polyester, or acrylic material, but is not limited thereto.

Ester compounds obtained by the method of purifying terephthalate-based ester compounds may have a concentration of impurities ranging from several ppm to several hundred ppm.

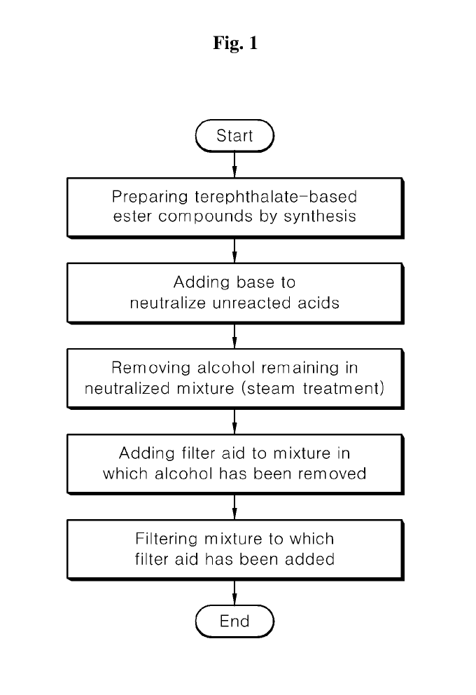

FIG. 1 is a schematic diagram illustrating a method of purifying terephthalate-based ester compounds according to one embodiment of the present invention.

Hereinafter, specific examples of the present disclosure will be described. It is to be understood, however, that the examples described below are only for illustrative purposes or to illustrate the present disclosure, and the present disclosure should not be construed as being limited thereto.

### EXAMPLES

### Example 1

350 g (2.107 moles) of terephthalic acid, 687 g (5.28 moles) of 2-ethylhexanol and 0.208 g (200 ppm) of tetraisopropoxide were added to a 2 L round bottom flask equipped with a stirrer, thermometer, heating mantle and distillation column. Upon heating, the reaction initiated at about 180° C. As the reaction progressed, produced water was generated and the produced water was removed by the distillation column. After completion of the reaction, aqueous Na₂CO₃ solution was added to neutralize unreacted terephthalates, and steam was added at 180° C. to remove the remaining 2-ethylhexanol. N₂ was then injected to remove a small amount of water, and vigorously stirred with 5 g of diatomite, followed by filtration with a vacuum filter. The filtration rate was measured in the amount of filtration per hour (g/hr). The diatomite used had pH 10 and included 90 to 100 parts by weight of SiO₂, 1 to 10 parts by weight of Al₂O₃, 1 to 10 parts by weight of Fe₂O₃, 0.1 to 1 part by weight of CaO, 0.1 to 1 part by weight of MgO, and water. The diatomite had an average particle size of 48.0 μm.

### Example 2

A terephthalate-based ester compound was synthesized in the same manner as in Example 1 and purified, using diatomite having the same composition as that of the diatomite used in Example 1, except that the diatomite particles having an average particle size of 51.0 μm was used.

### Example 3

A terephthalate-based ester compound was synthesized in the same manner as in Example 1 and purified, using diatomite having the same composition as that of the diatomite used in Example 1, except that the diatomite particles having an average particle size of 53.1 μm was used.

### Example 4

A terephthalate-based ester compound was synthesized in the same manner as in Example 1 and purified, using diatomite having the same composition as that of the diatomite used in Example 1, except that the diatomite particles having an average particle size of 85.2 μm was used.

### Comparative Example 1

A terephthalate-based ester compound was synthesized in the same manner as in Example 1 and purified, using diatomite having the same composition as that of the diatomite used in Example 1, except that the diatomite particles having an average particle size of 38.9 μm was used.

### Comparative Example 2

A terephthalate-based ester compound was synthesized in the same manner as in Example 1 and purified, using diatomite having the same composition as that of the diatomite used in Example 1, except that the diatomite particles having an average particle size of 28.0 μm was used.

The average size and the filtration rate of the diatomite particles added in the filtration process during the process of purifying the ester compounds in Examples 1 to 4 and Comparative Examples 1 and 2 are listed in Table 1 below. The filtration rate was measured three times in each of Examples 1 to 4 and Comparative Examples 1 and 2, and the average value was described. In addition, the filtration rate increased/decreased percentage (%) was calculated relative to Comparative Example 1 and listed in Table 1 below.

The results are summarized in Table 1 below.

TABLE 1

| Division | Ex. 1 | Ex. 2 | Ex. 3 | Ex. 4 | C. Ex. 1 | C. Ex. 2 |
|---|---|---|---|---|---|---|
| Average diameter of | 48.0. | 51.0. | 53.1. | 85.2. | 38.9. | 28.0. |
| diatomite particles | | | | | | |
| added (μm) | | | | | | |
| Filtration rate (g/hr) | 69.0. | 70.7. | 86.5. | 71.0. | 63.9. | 66.0. |
| Filtration rate | 8.0. | 10.6. | 35.4. | 11.1. | — | 3.3. |
| increased/decreased | | | | | | |
| percentage (%) | | | | | | |

From the results shown in Table 1, the filtration rate of the filter in Examples 1 to 4 was improved in comparison with that of Comparative Examples 1 and 2.

While the present disclosure has been particularly shown and described with reference to exemplary embodiments thereof, it is to be understood by those skilled in the art that the disclosure is not limited to the embodiments and various changes in form and details may be made therein without departing from the spirit and scope of the invention as defined by the appended claims.

What is claimed is:

1. A method of purifying terephthalate-based ester compounds, comprising:
adding a base to a resultant reaction mixture comprising terephthalate-based ester compounds synthesized by esterification of terephthalic acid and alcohol so as to neutralize unreacted acids;
applying steam to the resultant reaction mixture which has been neutralized;
adding a filter aid to the resultant reaction mixture which has been steamed prior to filtration through a filter; and
filtering through a filter the resultant reaction mixture to which the filter aid has been added,
wherein the filter aid comprises diatomite particles having an average particle size of 50 μm to 90 μm.

2. The method according to claim 1, wherein the alcohol is an alkanol having a linear or branched (C₄-C₁₃) alkyl group.

3. The method according to claim 1, wherein the alcohol comprises at least one selected from the group consisting of butanol, 2-ethylhexanol, isononyl alcohol, isodecyl alcohol, propyl heptanol, and combinations thereof.

4. The method according to claim 1, wherein the base comprises at least one selected from the group consisting of sodium hydroxide (NaOH), sodium carbonate (Na₂CO₃), sodium bicarbonate (NaHCO₃), and combinations thereof.

5. The method according to claim 1, wherein the steam is at a temperature of 140 to 200° C. and under a pressure of 2 bar to 10 bar.

6. The method according to claim 1, wherein the diatomite has a pH of 7 to 10 and comprises 90 to 100 parts by weight of SiO₂, 1 to 10 parts by weight of Al₂O₃, 1 to 10 parts by weight of Fe₂O₃, 0.1 to 1 part by weight of CaO, 0.1 to 1 part by weight of MgO, and water.

7. The method according to claim 1, wherein the diatomite is added in an amount of 1 to 10 parts by weight based on 100 parts by weight of the resultant reaction mixture which has been steamed.

8. The method according to claim 1, wherein the filter is a filter membrane on which a precoat layer containing diatomite particles is formed.

9. The method according to claim 1, wherein the method removes neutralized salts of unreacted terephthalates.

10. The method according to claim 1, wherein the method is a rinsing-free process.
